(19) United States
(12) Patent Application Publication
(10) Pub. No.: US 2026/0102284 A1
(43) Pub. Date: Apr. 16, 2026

(54) OPHTHALMIC TOOL FOR AB-INTERNO REMOVAL OF OPHTHALMIC TISSUE AND METHOD THEREFOR

(71) Applicant: MOR RESEARCH APPLICATIONS LTD., Ramat Gan (IL)

(72) Inventors: Avner BELKIN, Ramat Gan (IL); Yoram SHMUELI, Ramat Gan (IL)

(21) Appl. No.: 18/855,342

(22) PCT Filed: Apr. 9, 2023
(86) PCT No.: PCT/IB2023/053613

§ 371 Date: Oct. 9, 2024

Related U.S. Application Data
(60) Provisional application No. 63/329,454, filed on Apr. 10, 2022.

(51) Int. Cl. A61F 9/007; A61B 17/00; A61B 17/3207

(52) U.S. Cl. CPC A61F 9/00781; A61B 2017/00367; A61B 17/320708

(57) ABSTRACT

Embodiments pertain to an ophthalmic tool for ab-interno removal of ophthalmic tissue from a human eye, the human eye including trabecular meshwork tissue and a Schlemm's canal. The tool comprises an excise device having a longitudinal centerline, and shaped and dimensioned pull-string for snug insertion into the Schlemm's canal, and has a leading nose, and a trailing knife support with a pair of spaced apart longitudinally directed knife blades mounted upright on the knife support.

### CROSS-REFERENCE TO RELATED APPLICATIONS

This application claims domestic benefit and international priority to U.S. Patent Application Ser. No. 63/329,454, filed on Apr. 10, 2022, and which is incorporated by reference herein in its entirety.

### TECHNICAL FIELD

The invention relates to ophthalmic tools for ab-interno removal of ophthalmic tissue and a surgical method therefor.

### BACKGROUND

Glaucoma is the leading cause of preventable irreversible blindness worldwide and intricately linked to raised intraocular pressure (IOP). A human eye has an anterior chamber with an outermost anterior chamber angle towards the eye's sclera. A thin strip of tissue called the trabecular meshwork (TM) runs circumferentially in the anterior chamber angle. As viewed in dorsal direction, the Schlemm's Canal (SC) circumferentially overlies the trabecular meshwork. In a healthy human eye, aqueous humor which is produced behind an iris in the posterior chamber flows through the pupil into the anterior chamber and is drained into the venous system through the following structures, in what is termed the conventional outflow system: trabecular meshwork, Schlemm's canal, collector channels, aqueous veins and conjunctival veins. In a human eye suffering from glaucoma, drainage of aqueous humor is impaired by elevated flow resistance through the trabecular meshwork, thereby resulting in an increased IOP.

There are two main approaches to the surgical treatment of glaucoma, namely, bypassing the physiological drainage system or trying to enhance flow through it. In the latter approach, there are technologies for stenting a Schlemm's canal, dilating it, or cutting it from an anterior chamber into a Schlemm's canal as in goniotomy or from inside a Schlemm's canal in a so-called Gonioscopy Assisted Transluminal Trabeculotomy (GATT) procedure. GATT is an effective IOP lowering procedure. However, since tissue is not removed from the eye, there is potential for scarring, fibrosis, reclosure of the cleft that was opened and obstruction to flow. There is also an option to remove a strip of trabecular meshwork though only for up to one third of its circumference using a so-called Kahook dual blade. Also, use of the Kahook dual blade is from an anterior chamber into a Schlemm's canal making control of the exact location challenging.

There is a need for ophthalmic tools for improved lowering of a raised IOP, and method therefor.

The description above is presented as a general overview of related art in this field and should not be construed as an admission that any of the information it contains constitutes prior art against the present patent application.

### BRIEF DESCRIPTION OF DRAWINGS

In order to understand embodiments of the invention and to see how it can be carried out in practice, example embodiments will now be described, by way of non-limiting examples only, with reference to the accompanying drawings in which similar parts are likewise numbered, and in which:

FIG. 1 is a schematic view of an ophthalmic tool including an excise device and a flexible pullstring for ab-interno removal of ophthalmic tissue from a human eye, according to some embodiments.

FIG. 2 is a schematic close-up view of the excise device, according to some embodiments.

FIG. 3 is a schematic back side view of the excise device, according to some embodiments.

FIG. 4 is a schematic partial para-sagittal cross section of a human eye of a supine human subject before ab-interno removal of ophthalmic tissue, according to some embodiments.

FIG. 5 is a schematic coronal view of a human eye prepared with a single corneal aperture for deployment of the ophthalmic tool for ab-interno removal of ophthalmic tissue, according to some embodiments.

FIG. 6 is a schematic top plan view of the human eye showing insertion of the ophthalmic tool's pullstring free end thereinto through the single corneal aperture, according to some embodiments.

FIG. 7 is a schematic coronal cross-section of the human eye with the pullstring placed through the same incision into the Schlemm's canal, according to some embodiments.

FIG. 8 is a schematic view showing the pull string inserted into the anterior chamber, passed 360 through Schlemm's canal, and extracted through the same corneal incision aperture before insertion of the excise device into the human eye through the single corneal aperture, according to some embodiments.

FIG. 9 is a schematic top plan view of a human eye prepared with a pair of spaced apart corneal apertures for the deployment of the ophthalmic tool for ab-interno removal of ophthalmic tissue, according to some embodiments.

FIG. 10 is a schematic top plan view of the human eye showing the insertion of the ophthalmic tool's pullstring free end thereinto through an entry corneal aperture, according to some embodiments.

FIG. 11 is a schematic coronal cross section of the human eye with the pullstring placed in the Schlemm's canal, inserted and extracted through different corneal incision apertures, according to some embodiments.

FIG. 12 is a schematic top plan view of the human eye showing removal of the ophthalmic tool's pullstring free end from the human eye through a second, spaced apart exit corneal aperture before insertion of the excise device into the human eye through the single corneal aperture, according to some embodiments.

FIG. 13 is a schematic partial parasagittal cross-section of the human eye with the excise device placed in the Schlemm's canal, according to some embodiments.

FIG. 14 is a schematic pictorial view showing the excise device excising ophthalmic tissue on clockwise dialing around the Schlemm's canal, according to some embodiments.

FIG. 15 is a schematic partial para-sagittal cross-section of the human eye after ab-interno removal of ophthalmic tissue, according to some embodiments.

FIG. 16 is a flow diagram of a surgical method for ab-interno removal of ophthalmic tissue, according to some embodiments.

### DETAILED DESCRIPTION

Embodiments of the present invention is directed towards an ophthalmic tool for ab-interno removal of trabecular meshwork and a surgical method therefor.

The ophthalmic tool includes an excise device and a flexible pullstring for pulling the excise device circumferentially around a Schlemm's canal. The excise device includes a leading nose and a trailing knife support relative to a direction of pulling around a Schlemm's canal. The excise device includes a pair of spaced apart knife blades with knife edges for excising ophthalmic tissue as it is pulled or pushed circumferentially around a Schlemm's canal by its pullstring.

In some embodiments, the excise device includes a pair of spaced apart knife blades extending from the connecting base to form a U-shaped excise device. In some examples, the connecting base may also function or be configured as a knife blade. In some examples, the heights of the pair of spaced apart knife blades may be identical or different. In some embodiments, the U-shaped excise device may be configured for allowing pulling or pushing the U-shaped excise device along a patient's Schlemm's canal.

Although embodiment disclosed herein may pertain to a configuration where the excise device is pulled along a patient's Schlemm's canal, this should not be construed in a limiting. Accordingly, the excise device may additionally or alternatively include configurations allowing pushing of the excise device along the patient's Schlemm's canal for excising, at least a portion or the entirety of the trabecular meshwork tissue, possibly along with some of the Schlemm's canal.

The ophthalmic tool is manufactured from suitable bio-compatible materials including, for example, metallic and/or non-metallic material, inter alia stainless steel, ceramics, plastics, and the like. The ophthalmic tool can be provided as either a discrete excise device and a discrete flexible or a single monolithic construction. In the former case, the ophthalmic tool's components can be provided as a ready-for-use assembly. Alternatively, the ophthalmic tool's components can be provided separately and assembled by a surgeon during an ophthalmic procedure. Commercially available suture wire can be readily employed for the pullstring.

In some embodiments, the surgical method includes an initial step of preparing a human eye with at least one corneal incision. The at least one corneal incision is, for example, a self-sealing corneal incision. The surgical method includes a subsequent step of threading an ophthalmic tool's pullstring circumferentially around a Schlemm's canal before removal of ophthalmic tissue. The surgical method includes a subsequent step of removal of ophthalmic tissue including removal of at least trabecular meshwork tissue and possibly inner wall of Schlemm's canal for improving efficacy of IOP lowering. The excise device can be optionally angled to also remove an inner wall of a Schlemm's canal for leaving a circumferential cleft between a trabecular meshwork tissue's remaining edge and a Schlemm canal's inner wall.

The surgical method facilitates full circumferential removal of trabecular meshwork tissue and a Schlemm's canal's inner wall thereby maximizing potential drainage area of aqueous humor. In summary, the ophthalmic tool affords an exact, safe and reliable medical procedure for efficient IOP lowering.

### Ophthalmic Tool

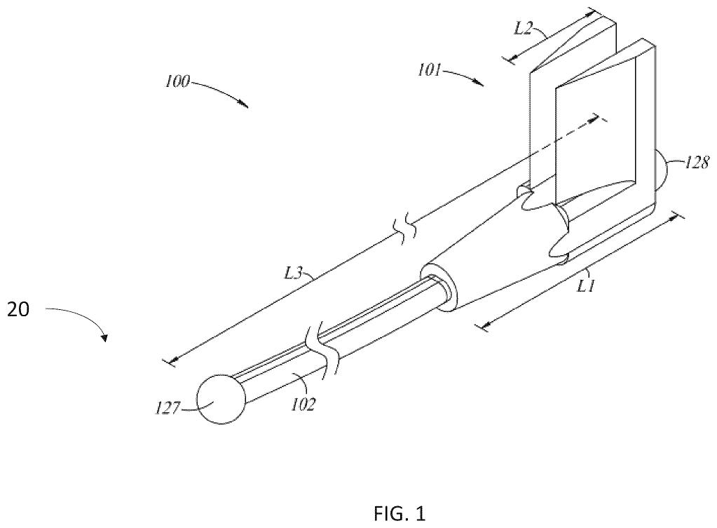

FIG. 1 to FIG. 3 show an ophthalmic tool 100 for performing an ab-interno removal of ophthalmic tissue. The ophthalmic tool 100 includes a discrete excise device 101 shaped and dimensioned for snug insertion into a Schlemm's canal and a discrete flexible pullstring 102 for pulling the excise device 101 around a Schlemm's canal about an intended direction of travel X. Alternatively, the ophthalmic tool 100 can be supplied as a monolithic construction. In some examples, the term “snug insertion” refers to an excise device configuration that allows form-fittingly engaging the excise device 101 into the Schlemm's canal with respect to a desired roll orientation of the excise device.

The excise device 101 has a longitudinal centerline or axis 103 extending along the intended direction of travel X, and includes a leading nose 104 and a trailing knife support 106. The terms “leading” and “trailing” are defined relative to the intended direction of travel X of the excise device 101 around a Schlemm's canal. The excise device 101 can be equally dialed clockwise or counterclockwise around a Schlemm's canal depending on a surgeon's preference.

The nose 104 has, for example, a truncated, straight or curved, conical or frustrum shape including a nose leading face 107 and a nose peripheral face 108. In some examples, the nose peripheral face 108 in the intended direction of travel X at a convergence angle ranging, for example, from about 5° to 15°, including, e.g., 10°. In some cases, the nose 104 can also have a non-circular (e.g., polygonal) base face.

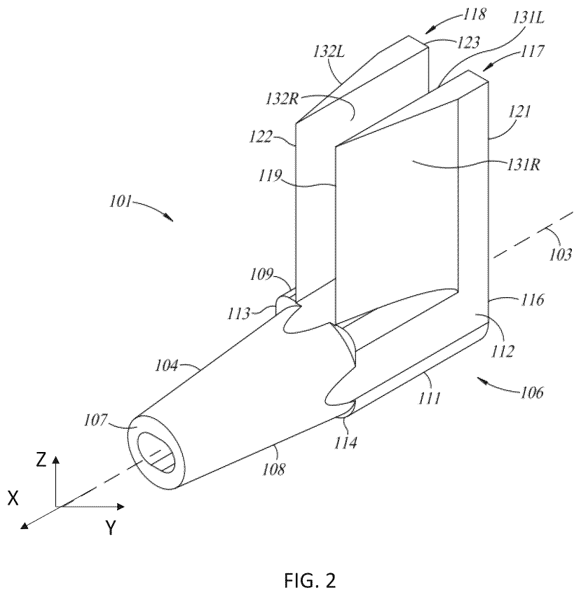

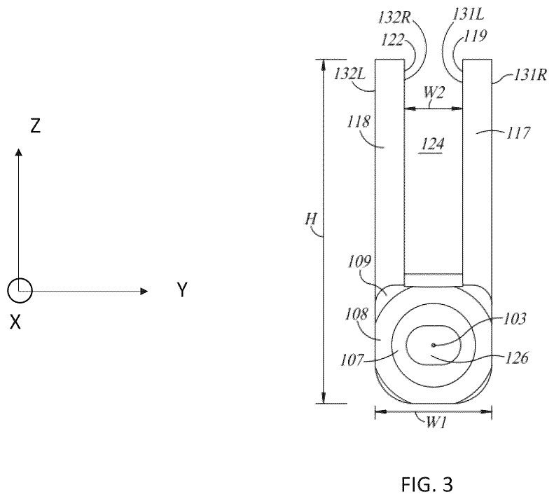

As shown in FIGS. 2 and 3, the knife support 106 may have a rectangular parallelepiped cross-sectional shape including a knife support top face 109, optionally possibly also slightly sloped in a direction approaching the meshwork when device is operably engaged with ophthalmic tissue, a knife support bottom face 111, a knife support right face 112, a knife support left face 113, a knife support front end face 114 and a knife support rear end face 116. The terms “left”, “right”, “rear” and “front”, as used herein pertain to excise device elements or features as viewed from a direction opposite to the ophthalmic tool's travel direction x.

The knife support 106 includes a pair of spaced apart knife blades 117 and 118 mounted upright on the knife support top face 109 co-directional with the longitudinal centerline 103. In one example, the two knife blades 117 and 118 may have equal shapes and/or dimensions. In another example, the two knife blades 117 and 118 may have different shapes and/or dimensions.

Optionally, the knife blade 117 extends upwards from the knife support right face 112 and the knife blade 118 extends upwards from the knife support left face 113. Optionally, the knife blade 117 and knife blade 118 can be straight blades or arcuate blades. Optionally, the knife blade 117 and the knife blade 118 are wedge shaped such that the knife blade 117 has a leading knife edge 119 and a trailing knife face 121 and the knife blade 118 has a leading knife edge 122 and a trailing knife face 123.

In some examples, the knife support 106 also includes a support knife blade (not shown) having a tapering surface extending in X-direction to terminate in a leading support knife edge (not shown) for cutting along the same travel direction as the spaced apart knife blades 117 and 118. Optionally, the support knife edge may extend between knife edge 122 and knife edge 119. Optionally, the support knife edge may be offset in the travel direction relative to the knife edge 122 and knife edge 119.

Optionally, both the leading knife edges 119, 122, and trailing knife faces 121, 123, extend away from knife top support face 109 spanned by X- and Y-axes, along Z-direction, of a Cartesian coordinate system schematically shown in the Figures. In some examples, one or both leading knife edges 119, 122 may be concave or convex-shaped, with respect to the X-direction. Additional or alternative knife shapes may be conceived.

The knife blade 117 and the knife blade 118 bound an excise device channel 124 therebetween co-extensive with the knife support top face 109 spanned by X- and Y-axes, and transverse to the longitudinal centerline 103. The leading knife edge 119 and the leading knife edge 122 are intended to cut opposite sides of ophthalmic tissue as the excise device 101 is pulled along a Schlemm's channel to form a strip of ophthalmic tissue which extends in a rearward direction through the excise device channel 124.

As schematically shown in FIG. 3, the right blade 117 has a left knife surface 131L and a right knife surface 131R extending from its leading knife edge 119 to opposing edges of its trailing knife face 121 and the left blade 118 has a left knife surface 132L and a right knife surface 132R extending from its leading knife edge 122 to opposing edges of its trailing knife face 123 with respect to a viewing direction opposite to the direction of travel X. The left knife surface 131L and the right knife surface 132R form the corresponding left and right surfaces of the excise device channel 124 with respect to a viewing direction opposite to the direction of travel X.

In some examples, the knife surfaces 131 and 132 may be straight surfaces or curved surfaces.

In some examples, the knife surfaces 131 and 132 may be parallel and non-parallel surfaces.

In some examples, the knife surfaces 131 and 132 may be curved to have common center of curvature.

In some examples, the knife surfaces 131 and 132 may be curved to have a common center of curvature.

The knife surfaces 131 and 132 may have various shapes and/or orientations relative to each other.

In some examples, one or both the knife surfaces 131 and 132 and/or corresponding leading edges may be curved or have an orientation relative to knife support top face 109 to approach each other in Z-direction.

In some examples, one or both the knife surfaces 131 and 132 and/or corresponding leading edges may be curved or have an orientation relative to each other to diverge from each other in Z-direction.

In some examples, one or both the knife surfaces 131 and 132 and/or corresponding leading edges may be angled or titled in counterclockwise or clockwise direction with respect to a viewing direction opposite to the direction of travel X.

In some examples, one or both the knife surfaces 131 and 132 and/or corresponding leading edges may be curved or have an orientation relative to the knife support top face 109 to approach each other in Z-direction.

In some examples, one or both the knife surfaces 131 and 132 and/or corresponding leading edges may extend perpendicularly from knife support top face 109 and be parallel relative to each other.

In some examples, the knife surfaces 131 and 132 may be parallel relative to each other, or have an orientation to approach or diverge from each other in X-direction.

The excise device 101 includes a longitudinally directed throughgoing bore 126 optionally centered along the longitudinal centerline 103. The throughgoing bore 126 may have a non-circular cross section transverse to the longitudinal centerline 103, which may conform with the non-circular cross-section of the pullstring. The throughgoing bore 126 and the pullstring 102 may have, for example, corresponding oval shaped, polygonal shaped, and/or have otherwise non-circular cross-sectional geometry, e.g., to prevent roll of the excise device 101 about the pullstring 102 while pulling the device circumferentially. In addition, this may allow the practitioner to control the roll orientation of the excise device 101 relative to the meshwork.

The excise device 101 has, for example, the following approximate dimensions:

| | |
|---|---|
| Excise device length | L1 = 1 mm-1.5, (e.g., 1.1. mm) |
| Excise device width | W1 = 0.2-0.5 mm (e.g., 0.3 mm) |
| Excise device height | H = 0.5-1 mm (e.g., 0.75 mm) |
| Knife blade length | L2 = 0.2-0.75 mm (e.g., 0.5 mm) |
| Excise device channel width | W2 = 0.1-0.3 mm 9 e.g., 0.15 mm) |
| Bore width | 0.1-0.18 mm (e.g., 0.14 mm) |
| Bore height | 0.05-0.15 mm (e.g., 0.10 mm) |

The pullstring 102 has two opposite ends: a pullstring free end 127 and a pullstring stop end 128 opposite the pullstring free end 127. The pullstring 102 has a transverse cross section corresponding to the throughgoing bore 126 for ensuring, with respect to the longitudinal centerline 103, correct or desired roll orientation of the ophthalmic tool 100 in a Schlemm's canal and the correct orientation is maintained to allow performing the ophthalmic procedure.

In some examples, the pullstring free end 127 is enlarged for assisting to thread the pullstring 102 circumferentially around a Schlemm's canal. In some examples, the pullstring stop end 128 is enlarged relative to the throughgoing bore 126 such that it stops thereagainst on threading the pullstring 102 therethrough.

The pullstring 102 has a sufficient length L3 of, for example, at least 20 cm to enable a surgeon to fully thread the pullstring 102 around a Schlemm's canal and have a leading section exterior to the human eye for subsequent manipulation.

### Surgical Method of Ab-Interno Removal of Ophthalmic Tissue

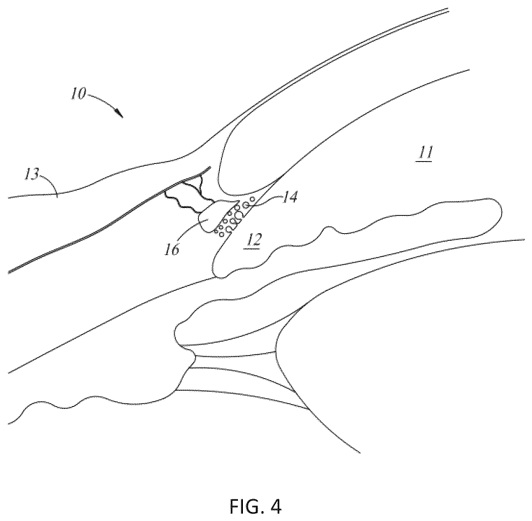

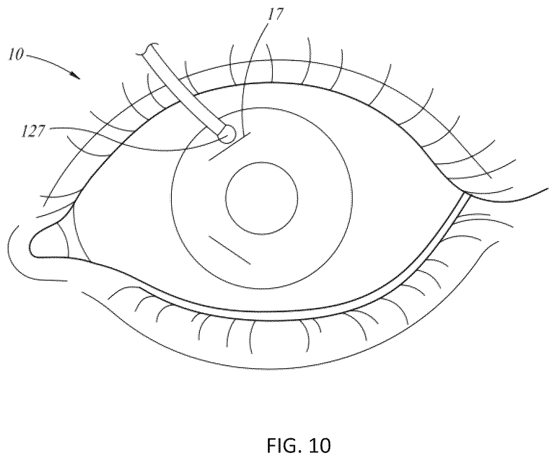

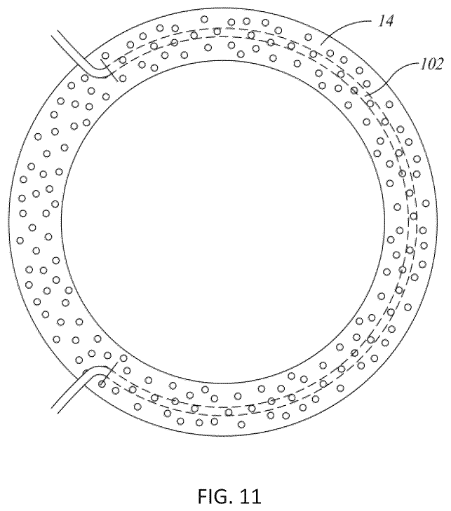

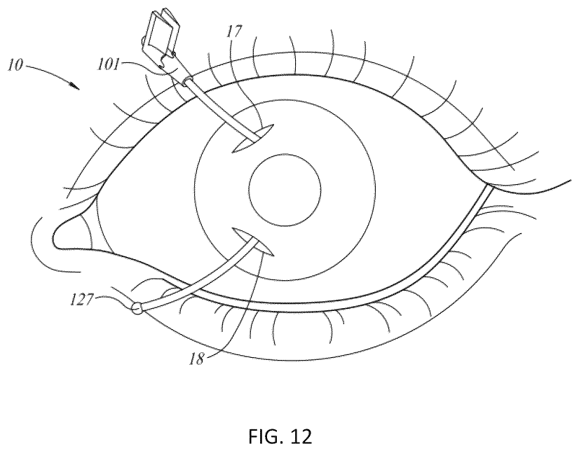

The treatment method of ab-interno removal of trabecular meshwork is now described with reference to FIG. 4 to FIG. 16 with regards to a human subject in a supine position.

FIG. 4 schematically shows a human eye 10 having an anterior chamber 11 with an outermost anterior chamber angle 12 towards the eye's sclera 13, trabecular meshwork 14 tissue running circumferentially in the anterior chamber angle 12 and a Schlemm's canal 16 overlying the trabecular meshwork 14, as viewed in from a dorsal direction. The trabecular meshwork tissue 14 and the Schlemm's canal 16 have approximate circumferences of about 6 cm along.

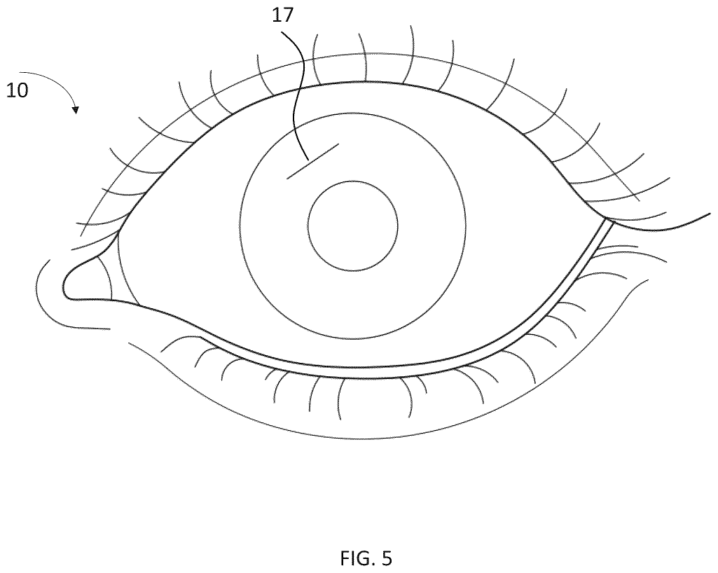

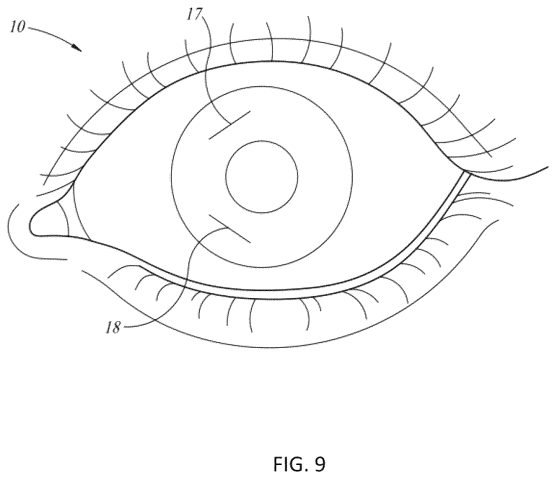

The surgical method can be performed through either a single corneal aperture (see FIGS. 5 to 8) or a pair of corneal apertures spaced apart, for example, by about 4-8 mm (see FIGS. 9 to 12). The corneal apertures are, for example, self-sealing corneal incisions or paracenteses towards an anterior chamber 11's nasal aspect.

In the case of a discrete excise device 101 and a discrete pullstring 102, a surgeon may assemble the ophthalmic tool 100 before operating on a subject. The non-circular cross section of the excise device's throughgoing bore 126 and correspondingly non-circular cross-sectional shape of the pullstring 102 allows for improved safety because it form-fittingly mandates the excise device 101 to be aimed at an anterior chamber and form-fittingly prevents its rotation about the travel axis during insertion into a Schlemm's canal and during its travel therearound.

The surgical method involves, for example, the following steps:

A surgeon prepares the human eye 10 as follows:

With reference now to FIGS. 5 to 8, FIG. 5 schematically shows making an aperture 17 towards the anterior chamber 11's nasal aspect. The surgeon may fill the human eye with a cohesive Ophthalmic Microsurgical Device (OVD). The surgeon may rotate the human eye 10 and employ a microscope for improved visualization of the anterior chamber angle 12. The surgeon uses an ophthalmic knife to incise the trabecular meshwork 14 at the anterior chamber angle's nasal aspect to expose the Schlemm's canal 16.

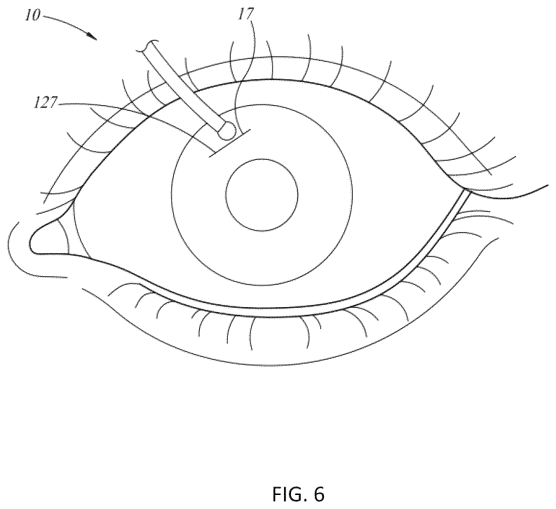

FIG. 6 schematically shows inserting the ophthalmic tool's pullstring free end 127 through the aperture 17 into the anterior chamber using ophthalmic forceps. The surgeon may use ophthalmic forceps to circumferentially thread the pullstring free end 127 around the Schlemm's canal 16. As the pullstring free end 127 extends around the Schlemm's canal 16, the excise device 101 approaches the aperture 17.

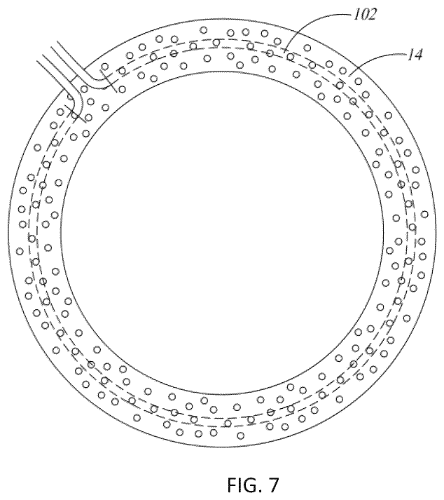

FIG. 7 shows the pullstring 102 circumferentially arranged around the Schlemm's canal 16 underneath trabecular meshwork tissue 14.

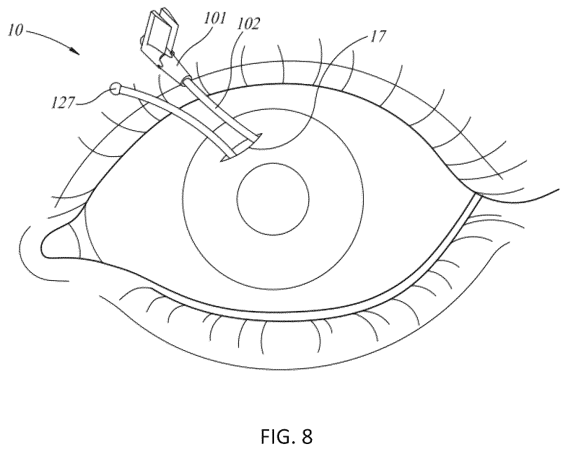

FIG. 8 schematically shows pulling of the pullstring free end 127 from the eye through the aperture 17 leaving a length of pullstring 102 circumferentially around the Schlemm's canal 16.

FIGS. 9 to 12 correspond to FIGS. 5 to 8 with regards to a spaced apart pair of corneal incisions 17 and 18 wherein the corneal incision 17 is employed as an entry corneal incision and the corneal incision 18 is employed as an exit corneal aperture.

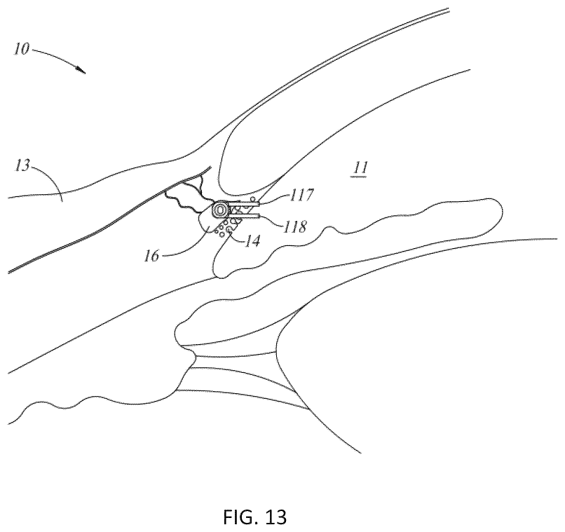

FIG. 13 schematically shows using ophthalmic forceps to snugly place the excise device 101 inside the Schlemm's canal 16 with its knife blade 117 and knife blade 118 extending towards the anterior chamber 11. FIG. 13 schematically shows that excise device 101 is angled or oriented to remove at least a portion of the entire width of the trabecular meshwork tissue 14 and the Schlemm canal's 16 inner wall. In some implementations, the excise device 101 can be angled or oriented relative to the Schlemm's canal 16 to remove trabecular meshwork tissue 14 only.

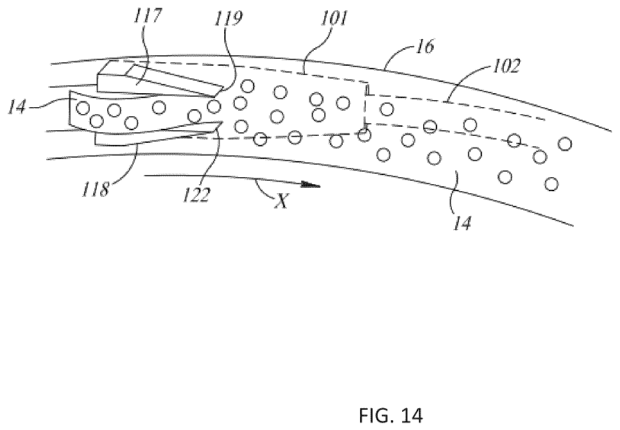

FIG. 14 schematically shows pulling the pullstring free end 127 to pull the excise device 101 circumferentially around the Schlemm's canal 16 in a clockwise travel direction X. The excise device's nose 104 raises the trabecular meshwork tissue 14 immediately before the knife support 106 ready for excising by the spaced apart knife edge 119 and knife edge 122. Circumferential passage of the excise device 101 around the Schlemm's canal 16 leads to a strip of trabecular meshwork tissue 14 and the Schlemm's canal's inner wall extending rearwards through the excise device channel 124 and outwardly beyond the excise device 101 in the opposite direction of the excise device 101's travel around the Schlemm's canal 16.

The surgeon pulls the pullstring free end 127 until the excise device 101 reaches the corneal aperture 17 in the case of a single corneal aperture and the exit corneal aperture 18 in the case of two corneal apertures 17 and 18. The surgeon uses ophthalmic forceps to remove the excise device 101 from the human eye 10.

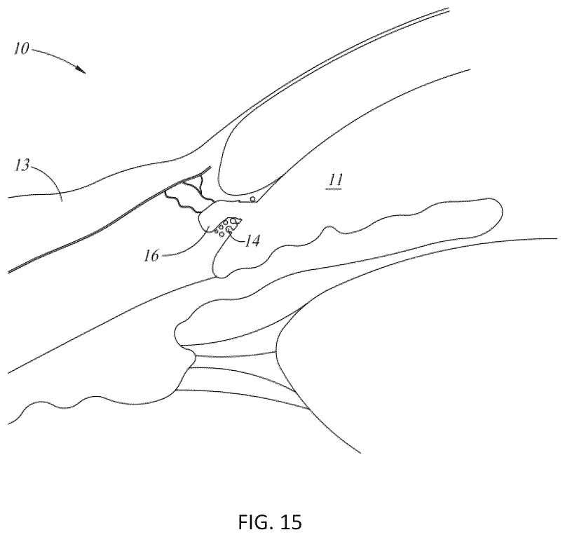

FIG. 15 schematically shows post-excision human eye with a cleft between trabecular meshwork tissue 14's remaining edge and the Schlemm's canal 16's inner wall.

The surgeon uses ophthalmic forceps to remove the excised strip of ophthalmic tissue. Depending on a single corneal aperture or a spaced apart pair of corneal apertures, the excise device 101 can excise nearly an entire circumference of trabecular meshwork tissue including, for example, at least 50%, at least 60%, at least 70%, at least 80%, at least 90% or 100% of the circumference. In some examples, at least 270°, at least 300°, at least 320°, at least 340°, or the entire circumference of the trabecular meshwork may be removed from a human eye.

The surgeon washes the anterior chamber 11 and closes the human eye 10 in a conventional manner.

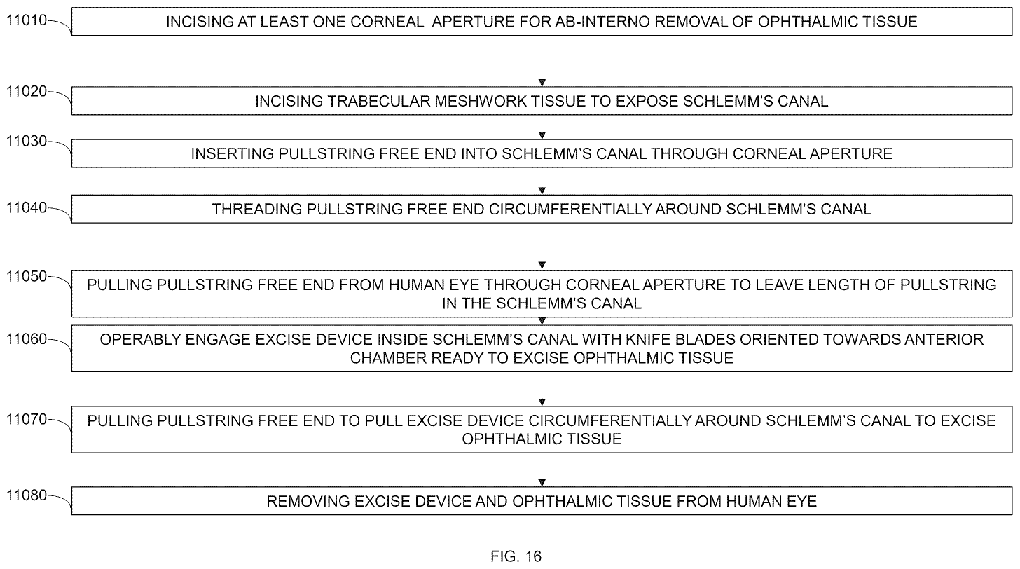

Additional reference is made to FIG. 16. In some embodiments, a method for ab-interno removal of trabecular meshwork may include, for example, preparing human eye for ab-interno removal of ophthalmic tissue (block 11010)

In some embodiments, the method may further include incising trabecular meshwork to expose underlying Schlemm's canal (block 11020).

In some embodiments, the method may include inserting a pullstring free end into the Schlemm's canal through a corneal aperture (block 11030).

In some embodiments, the method may further include threading the pullstring free end circumferentially around the Schlemm's canal (block 11040).

In some embodiments, the method may include pulling the pullstring free end from human eye through a corneal aperture to leave length of pullstring in the Schlemm's canal (block 11050).

In some embodiments, the method may further include operably engaging the excise device inside Schlemm's canal with knife blades oriented towards anterior chamber ready to excise ophthalmic tissue (block 11060).

In some embodiments, the method may include pulling the pullstring free end to pull excise device circumferentially around Schlemm's canal to excise ophthalmic tissue (block 11070).

In some embodiments, the method may further include removing the excise device, the pullstring and the strip of ophthalmic tissue from within the human eye (block 11080). This way, outflow of fluid from within the eye is improved, lowering intraocular pressure, reducing the risk or preventing irreversible blindness due to glaucoma.

### ADDITIONAL EXAMPLES

Example 1 pertains to an ophthalmic tool for ab-interno removal of ophthalmic tissue from a human eye, the ophthalmic tissue including trabecular meshwork tissue and a Schlemm's canal, the ophthalmic tool comprising:
an excise device having a longitudinal centerline, and shaped and dimensioned for snug insertion into the Schlemm's canal, the excise device having: a leading nose, and a trailing knife support with a pair of spaced apart longitudinally directed knife blades mounted upright on the knife support, the pair of spaced apart knife blades bounding an excise device channel therebetween transverse to the longitudinal centerline. Each knife blade may have a leading knife edge.

Example 2 includes the subject matter of example 1 and, optionally, wherein the excise device is configured for being circumferentially pushed (e.g., by a pushing rod), or pulled around the Schlemm's canal in a direction of travel such that when the ophthalmic tool is operably engaged with the human eye, the pair of spaced apart knife edges excises ophthalmic tissue including a strip of trabecular meshwork tissue.

Example 3 includes the subject matter of Examples 1 and/or 2 and, optionally, a flexible pullstring having a pullstring free end attachable to the excise device for pulling the excise device circumferentially around the Schlemm's canal in a direction of travel such that when the ophthalmic tool is operably engaged with the human eye, the pair of spaced apart knife edges excises ophthalmic tissue including a strip of trabecular meshwork tissue.

Example 4 includes the subject matter of any one or more of the examples 1 to 3 and, optionally, wherein the leading nose has a truncated generally conical shape converging in the direction of travel of the excise device circumferentially around the Schlemm's canal.

Example 5 includes the subject matter of any one or more of the examples 1 to 4 and, optionally, wherein the tool includes a discrete excise device and a discrete pullstring. Optionally, the discrete excise device is formed with a longitudinally directed throughgoing bore having a non-circular cross section transverse to the longitudinal centerline and has a trailing knife support face relative to the direction of travel. Optionally, the discrete pullstring includes a pullstring stop end opposite the pullstring free end, and the pullstring being shaped and dimensioned to snugly extend through the longitudinally directed throughgoing bore such that the pullstring stop end stops against the excise device's trailing knife support face.

Example 6 includes the subject matter of example 5 and, optionally, wherein the flexible pullstring has a non-circular cross-section corresponding to a non-circular cross-section of the throughgoing bore.

Example 7 includes the subject matter of any one or more of the examples 1 to 6 and, optionally, wherein the tool has a single monolithic construction.

Example 8 pertains to a flexible pullstring having a pullstring free attachable to an excise device for pulling the excise device circumferentially around the Schlemm's canal in a direction of travel such that when the ophthalmic tool is operably engaged with the human eye, a pair of spaced apart knife edges of the excise device excises ophthalmic tissue including a strip of trabecular meshwork tissue. Optionally, the flexible pullstring has a non-circular cross-section that conforms with a non-circular cross-section of a longitudinally extending throughgoing bore of the excise device.

Example 9 pertains to a kit for ab-interno removal of ophthalmic tissue from a human eye, the tissue including trabecular meshwork tissue, the kit comprising an excise device and a flexible pullstring, for example, according to any one or more of the preceding examples 1 to 8.

Example 10 pertains to a surgical method for ab-interno removal of ophthalmic tissue from a human eye, the human eye including trabecular meshwork tissue and a Schlemm's canal, the surgical method comprising the steps of:
providing an ophthalmic tool, for example, as described herein;
preparing at least one corneal aperture in the human eye;
incising the trabecular meshwork tissue;
inserting the ophthalmic tool's pullstring free end through a corneal aperture into the Schlemm's canal through the incised trabecular meshwork tissue;
threading the pullstring free end circumferentially around the Schlemm's canal;
removing the pullstring free end from the human eye through a corneal aperture to leave a length of pullstring in the Schlemm's canal;
inserting the excise device through a corneal aperture into the Schlemm's canal through the incised trabecular meshwork tissue;
pulling the pullstring free end to pull the excise device circumferentially around the Schlemm's canal to excise ophthalmic tissue including a strip of trabecular meshwork tissue;
removing the excise device from the human eye through a corneal aperture; and
removing the excised ophthalmic tissue from the human eye through a corneal aperture.

Example 11 includes the subject matter of example 10 and, optionally, wherein the step (b) includes preparing a single corneal aperture whereby the ophthalmic tool is inserted into the human eye and removed therefrom through the single corneal aperture.

Example 12 includes the subject matter of example 10 and, optionally, wherein the step (b) includes preparing a pair of spaced apart corneal apertures whereby the ophthalmic tool is inserted into the human eye through an entry corneal aperture and removed therefrom through an exit corneal aperture different from the entry corneal aperture.

In the discussion, unless otherwise stated, adjectives such as “substantially” and “about” that modify a condition or relationship characteristic of a feature or features of an embodiment of the invention, are to be understood to mean that the condition or characteristic is defined to within tolerances that are acceptable for operation of the embodiment for an application for which it is intended.

Unless otherwise specified, the terms ‘about’ and/or ‘close’ with respect to a magnitude or a numerical value may imply to be within an inclusive range of −10% to +10% of the respective magnitude or value.

“Coupled with” means indirectly or directly “coupled with”.

As used herein, the terms “proximal” and “distal” refer to a position closer to and away from a patient's body that would receive a port device.

The terms “longitudinal” and “axial” are interchangeable, and refer to an axis extending in the proximal and distal directions, unless otherwise expressly defined.

It should be noted that where an embodiment refers to a condition of “above a threshold”, this should not be construed as excluding an embodiment referring to a condition of “equal or above a threshold”. Analogously, where an embodiment refers to a condition “below a threshold”, this should not to be construed as excluding an embodiment referring to a condition “equal or below a threshold”. It is clear that should a condition be interpreted as being fulfilled if the value of a given parameter is above a threshold, then the same condition is considered as not being fulfilled if the value of the given parameter is equal or below the given threshold. Conversely, should a condition be interpreted as being fulfilled if the value of a given parameter is equal or above a threshold, then the same condition is considered as not being fulfilled if the value of the given parameter is below (and only below) the given threshold.

It should be understood that where the claims or specification refer to “a” or “an” element and/or feature, such reference is not to be construed as there being only one of that element. Hence, reference to “an element” or “at least one element” for instance may also encompass “one or more elements”.

As used herein the term “configuring” and/or ‘adapting’ for an objective, or a variation thereof, implies using materials and/or components in a manner designed for and/or implemented and/or operable or operative to achieve the objective.

Unless otherwise stated or applicable, the use of the expression “and/or” between the last two members of a list of options for selection indicates that a selection of one or more of the listed options is appropriate and may be made, and may be used interchangeably with the expressions “at least one of the following”, “any one of the following” or “one or more of the following”, followed by a listing of the various options.

As used herein, the phrase “A, B, C, or any combination of the aforesaid” should be interpreted as meaning all of the following: (i) A or B or C or any combination of A, B, and C, (ii) at least one of A, B, and C; and (iii) A, and/or B and/or C. This concept is illustrated for three elements (i.e., A, B, C), but extends to fewer and greater numbers of elements (e.g., A, B, C, D, etc.).

It is noted that the terms “operable to” or “operative to” can encompass the meaning of the term “adapted or configured to”. In other words, a machine “operable to” or “operative to” perform a task can in some embodiments, embrace a mere capability (e.g., “adapted”) to perform the function and, in some other embodiments, a machine that is actually made (e.g., “configured”) to perform the function.

Positional terms such as “upper”, “lower” “right”, “left”, “bottom”, “below”, “lowered”, “low”, “top”, “above”, “elevated”, “high”, “vertical” and “horizontal” as well as grammatical variations thereof as may be used herein do not necessarily indicate that, for example, a “bottom” component is below a “top” component, or that a component that is “below” is indeed “below” another component or that a component that is “above” is indeed “above” another component as such directions, components or both may be flipped, rotated, moved in space, placed in a diagonal orientation or position, placed horizontally or vertically, or similarly modified. Accordingly, it will be appreciated that the terms “bottom”, “below”, “top” and “above” may be used herein for exemplary purposes only, to illustrate the relative positioning or placement of certain components, to indicate a first and a second component or to do both.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

While the invention has been described with respect to a limited number of embodiments, it will be appreciated that many variations, modifications, and other applications of the invention can be made within the scope of the appended claims.

What is claimed is:

1. An ophthalmic tool for ab-interno removal of ophthalmic tissue from a human eye, the ophthalmic tissue including trabecular meshwork tissue and a Schlemm's canal, the ophthalmic tool comprising:
an excise device having a longitudinal centerline, and shaped and dimensioned for snug insertion into the Schlemm's canal,
the excise device having:
i) a leading nose, and
ii) a trailing knife support with a pair of spaced apart longitudinally directed knife blades mounted upright on the knife support,
the pair of spaced apart knife blades bounding an excise device channel therebetween transverse to the longitudinal centerline,
each knife blade having a leading knife edge.

2. The tool of claim 1, wherein the excise device is configured for being circumferentially pushed or pulled around the Schlemm's canal in a direction of travel such that when the ophthalmic tool is operably engaged with the human eye, the pair of spaced apart knife edges excises ophthalmic tissue including a strip of trabecular meshwork tissue.

3. The tool of claim 1, comprising a flexible pullstring having a pullstring free end attachable to the excise device for pulling the excise device circumferentially around the Schlemm's canal in a direction of travel such that when the ophthalmic tool is operably engaged with the human eye, the pair of spaced apart knife edges excises ophthalmic tissue including a strip of trabecular meshwork tissue.

4. The tool according to claim 1, wherein the leading nose has a truncated generally conical shape converging in the direction of travel of the excise device circumferentially around the Schlemm's canal.

5. The tool according to claim 1, wherein the tool includes a discrete excise device and a discrete pullstring,
wherein the discrete excise device is formed with a longitudinally directed throughgoing bore having a non-circular cross section transverse to the longitudinal centerline and has a trailing knife support face relative to the direction of travel, and
the discrete pullstring includes a pullstring stop end opposite the pullstring free end, and the pullstring being shaped and dimensioned to snugly extend through the longitudinally directed throughgoing bore such that the pullstring stop end stops against the excise device's trailing knife support face.

6. The tool of claim 5, wherein the flexible pullstring has a non-circular cross-section corresponding to a non-circular cross-section of the throughgoing bore.

7. The tool according to claim 1, wherein the tool has a single monolithic construction.

8. A flexible pullstring having a pullstring free attachable to an excise device for pulling the excise device circumferentially around the Schlemm's canal in a direction of travel such that when the ophthalmic tool is operably engaged with the human eye, a pair of spaced apart knife edges of the excise device excises ophthalmic tissue including a strip of trabecular meshwork tissue,
wherein the flexible pullstring has a non-circular cross-section that conforms with a non-circular cross-section of a longitudinally extending throughgoing bore of the excise device.

9. A kit for ab-interno removal of ophthalmic tissue from a human eye, the tissue including trabecular meshwork tissue, the kit comprising:
the excise device of claim 1 and a flexible pullstring.

10. A surgical method for ab-interno removal of ophthalmic tissue from a human eye, the human eye including trabecular meshwork tissue and a Schlemm's canal, the surgical method comprising the steps of:
a) providing the ophthalmic tool of claim 1;
b) preparing at least one corneal aperture in the human eye;
c) incising the trabecular meshwork tissue;
d) inserting the ophthalmic tool's pullstring free end through a corneal aperture into the Schlemm's canal through the incised trabecular meshwork tissue;
e) threading the pullstring free end circumferentially around the Schlemm's canal;
f) removing the pullstring free end from the human eye through a corneal aperture to leave a length of pullstring in the Schlemm's canal;
g) inserting the excise device through a corneal aperture into the Schlemm's canal through the incised trabecular meshwork tissue;
h) pulling the pullstring free end to pull the excise device circumferentially around the Schlemm's canal to excise ophthalmic tissue including a strip of trabecular meshwork tissue;
i) removing the excise device from the human eye through a corneal aperture; and
j) removing the excised ophthalmic tissue from the human eye through a corneal aperture.

11. The surgical method according to claim 10 wherein the step (b) includes preparing a single corneal aperture whereby the ophthalmic tool is inserted into the human eye and removed therefrom through the single corneal aperture.

12. The surgical method according to claim 10 wherein the step (b) includes preparing a pair of spaced apart corneal apertures whereby the ophthalmic tool is inserted into the human eye through an entry corneal aperture and removed therefrom through an exit corneal aperture different from the entry corneal aperture.

13. The method of claim 10, wherein the flexible pullstring has a pullstring free end attachable to the excise device for pulling the excise device circumferentially around the Schlemm's canal in a direction of travel such that when the ophthalmic tool is operably engaged with the human eye, the pair of spaced apart knife edges excises ophthalmic tissue including a strip of trabecular meshwork tissue.

14. The method of claim 10, wherein the tool includes a discrete excise device and a discrete pullstring,
wherein the discrete excise device is formed with a longitudinally directed throughgoing bore having a non-circular cross section transverse to the longitudinal centerline and has a trailing knife support face relative to the direction of travel, and
wherein the discrete pullstring includes a pullstring stop end opposite the pullstring free end, and the pullstring being shaped and dimensioned to snugly extend through the longitudinally directed throughgoing bore such that the pullstring stop end stops against the excise device's trailing knife support face.

15. The method of claim 10, wherein the flexible pullstring has a non-circular cross-section corresponding to a non-circular cross-section of the throughgoing bore.

16. The tool of claim 1, wherein one or both leading knife edges are concave-shaped or convex-shaped, with respect to a pulling direction.

17. The tool of claim 1, wherein one or both leading knife edges are straight blades.

18. A method for manufacturing the ophthalmic tool of claim 1, comprising providing the tool as a discrete excise device and a discrete flexible or a single monolithic construction.

19. The method for manufacturing of claim 18, comprising providing assemblable components for assembling the tool.

20. The method of claim 18, comprising manufacturing the tool from suitable bio-compatible materials.
